(19) United States
(12) Patent Application Publication
(10) Pub. No.: US 2005/0090427 A1
(43) Pub. Date: Apr. 28, 2005

(54) Delivery of drugs to the bloodstream in toothpaste

(76) Inventor: David W. Moskowitz, St. Louis, MO (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(21) Appl. No.: 10/969,629

(22) Filed: Oct. 20, 2004

Related U.S. Application Data
(60) Provisional application No. 60/512,540, filed on Oct. 20, 2003.

(51) Int. Cl. A61K 38/28; A61K 38/18; A61K 31/714; A61K 31/59

(52) U.S. Cl. 514/3; 514/12; 514/52; 514/167

(57) ABSTRACT

Methods are described for avoiding parenteral delivery of therapeutic compounds by forming an admixture of a therapeutic compound with an oral care composition to make the therapeutic compound available to the bloodstream of a subject in need of delivery of the therapeutic compound.

### CROSS-REFERENCE TO RELATED APPLICATIONS

This application claims priority to U.S. provisional patent application No. 60/512,540, filed Oct. 20, 2003.

### STATEMENT REGARDING FEDERALLY SPONSORED RESEARCH OR DEVELOPMENT

Not applicable.

### REFERENCE TO A SEQUENCE LISTING

Not applicable.

### BACKGROUND OF THE INVENTION

1. Field of the Invention

The present invention relates generally to drug delivery, and more particularly to drug delivery methods and compositions.

2. Description of the Related Art

Many drugs require parenteral administration because they are not otherwise accessible to the body through the more convenient oral administration route. For example, certain therapeutic peptides such as insulin, erythropoietin, and human growth hormone do not survive the acidic milieu of the stomach. Vitamin B12, which is used for example to treat patients with Cooley's anemia, requires stomach tissue factor to be absorbed through the stomach. To deliver such therapeutic compounds to the bloodstream, they are delivered by subcutaneous or intravenous injection. Subcutaneous injection is painful and can be difficult, especially for children. Regular subcutaneous injections of insulin are a particular problem for children suffering from type I diabetes mellitus, who often require multiple daily insulin injections. Accordingly, a need exists for improved methods of delivery of therapeutic compounds which are not readily bioavailable through oral administration and gastric absorption.

### SUMMARY OF THE INVENTION

The present invention encompasses a method for delivering a therapeutic compound to the blood stream of a subject in need of such drug delivery by admixing the therapeutic compound and an oral care composition. The therapeutic compound is, for example, in one embodiment selected from the group consisting of peptides. Such peptides include, for example, insulin, erythropoeitin and human growth hormone. In another embodiment of the method, the therapeutic compound is selected from the group consisting of vitamins otherwise requiring parenteral administration. Such vitamins include, for example, vitamin B12 and 1,25-dihydroxyvitamin D3.

In another embodiment, the invention encompasses a therapeutic composition comprising an admixture of an oral care composition and a therapeutic compound. The therapeutic compound is, for example, in one embodiment selected from the group consisting of peptides. Such peptides include, for example, insulin, erythropoeitin and human growth hormone. In another embodiment of the therapeutic composition, the therapeutic compound is selected from the group consisting of vitamins otherwise requiring parenteral administration. Such vitamins include, for example, vitamin B12 and 1,25-dihydroxyvitamin D3.

These and other features, aspects and advantages of the present invention will become better understood with reference to the following description, examples and appended claims.

### DETAILED DESCRIPTION OF THE PREFERRED EMBODIMENTS

Abbreviations and Definitions

To facilitate understanding of the invention, a number of terms and abbreviations as used herein are defined below as follows:

“oral care composition”: As used herein, “oral care composition” refers to a toothpaste, liquid toothpaste or a dental gel.

“therapeutic compound”: As used herein, the term “therapeutic compound” broadly refers to a pharmacologically active substance such as a bioactive molecule that is used for the treatment of a disease or condition in an animal. The term “therapeutic compound” encompasses but is not limited to bioactive molecules such as peptides which are not orally bioavailable, including insulin, erythropoietin, and human growth hormone, and also includes molecules such as vitamin B12 for patients such as those with Cooley's anemia, wherein the vitamin B12 requires stomach tissue factor for absorption into the bloodstream of the patient.

“therapeutic composition”: As used herein, the term “therapeutic composition” refers to an admixture of an oral care composition and a therapeutic compound.

Oral care compositions such as toothpastes and dental gels are well known for oral hygiene. Oral care compositions that include certain topical therapeutic compounds such local anaesthetics or topical antibiotics are known. U.S. Pat. No. 6,610,277 describes appetite suppressant toothpaste formulations which simultaneously promote oral hygiene and suppress appetite using appetite suppressing and depressing herbs such as guarana and green tea, which are orally bioavailable in the sense that they are absorbed through the stomach. However, oral compositions for the delivery of therapeutic compounds to the blood stream have not been described.

To practice the methods in accordance with the present invention, a therapeutic composition is made comprising an oral care composition in admixture with an amount of a therapeutic compound such that the resulting concentration of the therapeutic compound in the oral care composition produces an appropriate dosage of the therapeutic compound in a whole toothbrush head's worth of the oral care composition, or in a half toothbrush head's worth of the oral care composition. The amount of the therapeutic compound admixed with the oral care composition will vary with the exact therapeutic compound to be delivered, but amounts will generally be determined using appropriate dosages as provided in standard medical pharmacological textbooks such as GOODMAN & GILMAN'S THE PHARMACOLOGICAL BASIS OF THERAPEUTICS (Joel G. Hardman & Lee E. Limbird eds., 10^{th} ed. 2001). Dosage amounts and frequency for all compounds are in accordance with clinical practice for using the selected therapeutic compound in treating human adults or children as may be medically indicated. For example, insulin is used to treat adults and children with Type I diabetes mellitus. An exemplary therapeutic composition in accordance with the present invention contains an oral care composition and an amount of insulin. The insulin is, for example, a mixture of long-acting insulin and short-acting insulin (for example, 70% by weight long-acting insulin: 30% short-acting insulin). The insulin is admixed with the oral care composition in amount sufficient to obtain a resultant concentration of the insulin sufficient for delivery of recommended dosages of insulin in one, two, three or more applications of the oral care composition per day. For example, therapeutic compositions having various concentrations of insulin can be made, such as 10 units of insulin/toothbrush head worth of the therapeutic composition, 30 units of insulin/toothbrush head worth of the therapeutic composition, 70 units of insulin/toothbrush head worth of the therapeutic composition, and the like. Using a therapeutic composition with a known concentration of therapeutic compound, and thus known amount of therapeutic compound per toothbrush head worth (or per half toothbrush head worth) of the composition, the subject can brush one or more times per day to deliver the appropriate dosage of therapeutic compound (e.g. insulin) at the appropriate frequency. The same principles are used to determine amounts of other therapeutic compounds used in alternative formulations of the therapeutic compositions. The therapeutic compound is, for example, a peptide such as insulin, erythropoeitin or human growth hormone, or a vitamin otherwise requiring parenteral administration such as vitamin B12 and 1,25-dihydroxyvitamin D3. However, any therapeutic compound that can be stably admixed with an oral care composition and is preferably delivered to the bloodstream of a subject is used according to the methods and compositions of the present invention. For example, nitroglycerine, which is typically administered sublingually, can be advantageously incorporated in a therapeutic composition according to the methods and compositions of the present invention.

A standard oral care composition in accordance with the present invention is, for example, a toothpaste, liquid toothpaste or dental gel comprising from about 10% to about 30% vegetable glycerin; from about 10% to about 30% sorbitol as humectant; from about 10% to about 30% hydrated silica; from about 1% to about 30% purified water; from about 10% to about 30% xylitol; from about 1% to about 3% carrageenan; from about 1% to about 3% sodium lauryl sulfate; from about 1% to about 3% titanium dioxide; and from about to about 0.30%-1% oil of wintergreen. However, other oral care compositions as readily available and known in the art of dental hygiene can be used.

Generally, in a toothpaste, dental gel or liquid toothpaste, the humectant constitutes about 65% to about 75% by weight of the composition and the water content is about 10% to about 30% by weight. A gelling agent may be used in toothpastes or dental gels, such as natural and synthetic gums and gum-like materials, for example carrageenan, Irish moss, gum tragacanth, cellulose gums and synthetic silicated clays. The gelling agent constitutes about 1.0% to about 3.0% by weight of the oral care composition.

The oral care composition can contain a flavoring agent such as oil of wintergreen, peppermint, or the like, which may be combined with other flavor enhancing agents so that the resultant mixture has a pleasant taste and aftertaste.

The oral care composition will generally also include a dentally acceptable, substantially water insoluble polishing agent of the type commonly employed in toothpastes or dental gels. Such polishing agents include, for example, dicalcium phosphate, tricalcium phosphate, insoluble sodium metaphosphate, aluminum hydroxide, including hydrated alumina, colloidal silica, hydrated silica, precipitated silica and magnesium carbonate, calcium carbonate, calcium pyrophosphate, and bentonite, including suitable mixtures thereof. Silica-containing polishing agents can be used, such as amorphous hydrated silicon dioxide in the form of a white, odorless powder having an average particle size of 8 to 10 micrometers, amorphous silica (also called silica gel and silicic acid) as a dry white powder having an average particle size of 4 microns. A silica-containing polishing agent preferably constitutes about 10% to about 30% by weight of the oral care composition.

When the toothpaste is a visually clear gel or opacified gel, a polishing agent of colloidal silica, such as those sold under the trademark Syloid as Syloid 72 and Syloid 74 or under the trademark Santocel as Santocel 100 and synthetic alkali metal aluminosilicate complexes (including silica containing combined alumina) may be particularly suitable.

Organic surfactants are preferably used in the oral care composition of the present invention to assist in achieving thorough and complete dispersion of the compositions of the present invention throughout the oral cavity. The organic surfactant may be anionic or nonionic in nature. The surfactant is preferably a material with detergent properties which imparts to the composition detergent and foaming properties. Suitable anionic surfactants include water-soluble salts of higher fatty acid monoglyceride monosulfates, such as the sodium salt of the monosulfated monoglyceride of hydrogenated coconut oil fatty acids, higher alkyl sulfates, such as sodium lauryl sulfate, alkyl aryl sulfonates, such as sodium dodecyl benzene sulfonate, higher alkyl sulfoacetates, higher fatty acid esters of 1,2-dihydroxy propane sulfonates, olefin sulfonates and the like. Other particularly suitable surfactant materials include nonionic agents such as condensates of sorbitan monostearate with approximately 20 moles of ethylene oxide; condensates of ethylene oxide with propylene oxide; condensates of propylene glycol; condensation products of an alpha-olefin oxide containing 10 to 20 carbon atoms; a polyhydric alcohol containing 2 to 10 carbons; and 2 to 6 hydroxyl groups and either ethylene oxide or a heteric mixture of ethylene oxide and propylene oxide. The resultant detergents are heteric polymers having a molecular weight in the range of 400 to 1600 and containing 40% to 80% by weight of ethylene oxide, with an alpha-olefin oxide to polyhydric alcohol mole ratio in the range of 1:1 to 1:3. The amount of anionic or nonionic surfacant constitutes about 1% to about 3% by weight of the oral care composition.

The oral care composition of this invention may also contain conventional additional ingredients such as coloring or whitening agents and preservatives. These additional ingredients may each be added to the oral care composition in minimal amounts of up to about 5% by weight, and preferably up to 1%, provided they do not interfere with the therapeutic compound and compatibility properties of the finished product.

The oral care composition of this invention is prepared by conventional methods of making toothpaste, liquid toothpaste and dental gels. More specifically, for example, the gelling agent such as a cellulose gum is dispersed in glycerine, to which is added an aqueous solution containing the sweetening agent such as xylitol, followed by the addition of sorbitol and mixing for a period of about 20 minutes to hydrate the gum, mixing the gum mixture with the polishing agent in a mixer under a vacuum of 28-30 inches of pressure. Lastly, the flavor, the surfactant and therapeutic composition are added to a vacuum mixer, mixed for a period of about 15 minutes, and the resultant therapeutic composition is placed in a tube.

To use the resulting therapeutic composition in the practice of this invention to deliver the therapeutic compound to the bloodstream of a subject in need of such delivery, the therapeutic composition is applied regularly to the oral cavity by brushing the teeth at least once to three or more times a day as may be indicated by the dosage scheduling and concentration of the therapeutic compound in the therapeutic composition. Brushing is performed after or before meals, preferably after eating meals to simultaneously promote oral hygiene.

The oral care composition of the present invention comprises molecules of a sufficiently small size to readily pass through to the intravascular space, the barrier to which is temporarily disrupted through the act of brushing. Thus, the invention contemplates intravascular absorption of the therapeutic compound through surface blood vessels in the gums that are disrupted by the mechanical action of brushing. To increase vascular absorption of the therapeutic compound, a subject optionally flosses between teeth before applying the oral care composition, to further disrupt surface blood vessels in the subject's gums. The therapeutic compound is water soluble and absorbed into the subject's blood stream.

Other Embodiments

When introducing elements of the present invention or the preferred embodiments thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above constructions without departing from the scope of the invention, it is intended that all matter contained in the above description or shown in the accompanying drawings shall be interpreted as illustrative and not in a limiting sense. Indeed, various modifications of the invention in addition to those shown and described herein will become apparent to those skilled in the art from the foregoing description which do not depart from the spirit or scope of the present inventive discovery. Such modifications are also intended to fall within the scope of the appended claims.

References Cited

All publications, patents, patent applications and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention.

1. A method for delivering a therapeutic compound to the blood stream of a subject in need of such drug delivery by admixing the therapeutic compound and an oral care composition.

2. A method with claim wherein the therapeutic compound is selected from the group consisting of peptides.

3. A method in accordance with claim 2 wherein the therapeutic compound is selected from the group consisting of insulin, erythropoeitin and human growth hormone.

4. A method in accordance with claim 1 wherein the therapeutic compound is selected from the group consisting of vitamins otherwise requiring parenteral administration.

5. A method in accordance with claim 4 wherein the therapeutic compound is selected from the group consisting of vitamin B12 and 1,25-dihydroxyvitamin D3.

6. A therapeutic composition comprising an admixture of a oral care composition and a therapeutic compound.

7. A therapeutic composition in accordance with claim 6 wherein the therapeutic compound is selected from the group consisting of peptides.

8. A therapeutic composition in accordance with claim 7 wherein the therapeutic compound is selected from the group consisting of insulin, erythropoeitin and human growth hormone.

9. A therapeutic composition in accordance with claim 6 wherein the therapeutic compound is selected from the group consisting of vitamins otherwise requiring parenteral administration.

10. A therapeutic composition in accordance with claim 9 wherein the therapeutic compound is selected from the group consisting of vitamin B12 and 1,25-dihydroxyvitamin D3.
